(19) United States
(12) Patent Application Publication
(10) Pub. No.: US 2016/0060710 A1
(43) Pub. Date: Mar. 3, 2016

(54) COMPAS-PCR METHOD AND METHODS FOR DETECTING, IDENTIFYING OR MONITORING SALMONID SPECIES

(71) Applicant: NIVA, Oslo (NO)

(72) Inventor: Marc ANGLÈS D'AURIAC, Oslo (NO)

(73) Assignee: NIVA, Oslo (NO)

(21) Appl. No.: 14/783,068

(22) PCT Filed: Apr. 7, 2014
(86) PCT No.: PCT/NO2014/050049

§ 371 Date: Oct. 7, 2015

(30) Foreign Application Priority Data
Apr. 8, 2013 (NO) 20130468

(51) Int. Cl. C12Q 1/68

(52) U.S. Cl. CPC C12Q 1/6888; C12Q 1/686; C12Q 2600/156

(57) ABSTRACT

The present invention provides an asymmetric PCR method, the COMplementary-Primer-Asymmetric (COMPAS)-PCR, and specific methods for detecting, identifying or monitoring salmonid species. The present invention also encompasses oligonucleotide primers corresponding to species specific sequences. The use of the methods and primers are also an aspect of the present invention together with kits comprising said primers.

### FIELD OF THE INVENTION

The present invention provides an asymmetric PCR method, the COMplementary-Primer-Asymmetric (COMPAS)-PCR, and specific methods for detecting, identifying or monitoring salmonid species. The present invention also encompasses oligonucleotide primers corresponding to species specific sequences. The use of the methods and primers are also aspects of the present invention together with kits comprising said primers.

### BACKGROUND OF THE INVENTION

Since the Polymerase Chain Reaction (PCR) was invented by Kary Mullis in the mid-80s [1], Nucleic Acid (NA) amplification techniques have had an unprecedented development for molecular biology applications. A contributing factor to this success is its flexibility with the development of several modifications which expands the technical capabilities of PCR. In particular several methods have been developed for the detection of point mutations such as the Amplification Refractory Mutation System (ARMS) [2] and variants such as the PCR Amplification of Specific Allels (PASA) [3, 4], bidirectional-PASA [5] or Mistmatch Amplification Mutation Assay (MAMA) [6], Taq-MAMA [7] and Melt-MAMA [8]. Point mutations, also called Single Nucleotide Polymorphism (SNP), constitute differentiating genetic information which may be relevant in various contexts spanning from medical applications for disease diagnostics to population genetics and species identification. As the amount of PCR application increases, limitations inherent to DNA chemistry may become more challenging. For instance primer complementarity leading to primer dimer formation, is a limiting factor for the design of generic PCR.

It is believed that the asymmetric PCR method according to the present invention will alleviate PCR limitations due to primer complementarity incompatibility. Said method was further applied for the development of a PCR assay for direct repeat sequences.

NA technology can be characterized as noninvasive since very little subject sample is required for obtaining sufficient NA for analysis. The fish farming industry is in need for such noninvasive methods for the identification of salmonid species and in particular Salmo solar (Atlantic salmon) and Salmo trutta (brown trout) and their hybrids. Methods for species determination and inter-species hybridization between different salmonid fish species is an important tool in ecological studies, and when assessing the impact of aquaculture escapees on indigenous populations. Ecological studies have shown that inter species hybridization can severely impact population size and viability estimates. Morphological discrimination between hybrids and parental species is in many cases difficult (e.g. S. salar X S. trutta), and the behavior of the hybrids may be different from that of the parental species [9].

Identification of fish species by PCR has previously been addressed and solutions have been suggested. However, when selecting available methods for identification of salmonid species as e.g. in References [10-14], the method published by A. M. Pendas et al., (Chromosomal mapping and nucleotide sequence of two tandem repeats of Atlantic salmon 5 S rDNA Cytogenet Cell genet 67:31-36 (1994)) [14] seemed to give the best result among the methods tested. The inventor surprisingly discovered that said method was not reliable for the above mentioned purpose as the identification of the species Salmo salar and Salmo trutta were not distinguished satisfactorily using the PCR method and primers described in [14]. It is believed that the present invention provides a robust simple PCR method capable of specifically identify Salmo salar and preferably differentiate between Salmo Salar, Salmo trutta and their hybrids. The inventor tested the Salmo-A & B PCR method by A. M. Pendas et al.; the primers were originally designed based on the 5S rDNA sequence of the rainbow trout (Salmo gairdnerii, renamed Oncorhynchus mykiss) [15] and used on Salmo trutta and Salmo salar among other fish species [16]. These primers amplify 118 bp of the 120 bp coding sequence of the 5S rDNA together with the associated variable non coding sequence. The amplification product(s) varies in length and were used to differentiate close species. Two loci were originally found to be amplified for Salmo salar, one major product about 255 bp and a minor product around 525 bp. These results are corroborated by the 2 published sequences for Salmo salar gb S73107.1 & gb S73106.1. Similarly, Salmo trutta also showed a double band pattern but with longer products both for the major and minor loci [14]. Hybrids between Salmo salar and Salmo trutta were found to produce both product types for the major loci around 255 bp and longer respectively [16]. No product(s) difference between Salmo salar and for Salmo trutta was detected by the inventor using the method of A. M. Pendas et al., to differentiate Salmo salar from Salmo trutta and their hybrids. However, the genetic target of said PCR method, the 5S-rDNA, was used to develop the novel and inventive method together with specific oligonucleotide primers and COMPAS PCR method of the present invention.

### SUMMARY OF THE INVENTION

The present invention encompasses in a first aspect a method of asymmetric PCR comprising:
providing a nucleic acid sample to be used as a target template;
identifying target template(s)
performing a polymerase chain reaction, utilizing highly complementary primers wherein either the forward or the reverse primer concentration is decreased to unblock the PCR reaction by initially promoting linear amplification which will progressively shift towards exponential amplification by the COMplementary-Primer-ASymmetric (COMPAS)-PCR;
identifying the amplified nucleotide target sequence(s);

A second aspect of the present invention comprises a method for detecting, identification or monitoring salmonid species comprising:
providing a nucleic acid sample from salmonid to be used as (a) target template(s);
performing a polymerase chain reaction (PCR) applying COMplementary-Primer-ASymmetric (COMPAS)-PCR of the present invention, to amplify a nucleic acid target sequence of the template (s), utilizing a set or several sets of highly complementary primer pair(s) capable of priming said target(s);
identifying the amplified nucleotide target sequence(s);
determining the species.

In a third aspect the present invention provides oligonucleotide primers, selected from the oligonucleotides of Tables 1 and 2, or oligonucleotides with complementary sequences or functional equivalent sequences.

Further it is provided in a fourth aspect a kit for detecting and identification of salmonid species, comprising a collection of oligonucleotide primer pairs selected from Tables 1 and 2 in any combinations or complementary sequences thereof, capable of detecting salmonid species by the method of the present invention.

Finally in further aspects the present invention comprises use of the methods of the present invention. Use of oligonucleotide primers of the present invention or use of a kit for detecting and identification of salmonid species in accordance with the present invention.

Preferred embodiments are set forth in the dependent claims and in the detailed description of the invention

### DESCRIPTION OF THE FIGURES

FIG. 1. Illustrates COMPAS-PCR optimalisation example for Salmo salar & Salmo trutta, varying the concentration of the forward primer 5SNTS-23F from 0.6 to 0.05 μM while using a constant concentration of 0.6 μM for the reverse primer 5SNTS-22R+2 as described in Example 1.

FIG. 2. Illustrates a simplified diagram of the direct repeat-sequence COMPAS-PCR and in particular applied to SalmoID method.

FIG. 3. Illustrates a detailed diagram of the SalmoID method. All tested primers are shown by the arrows. “Salmo salar specific” shows the specific reverse primers with 3 nucleotide “overhang” in the NTS section.

FIG. 4. Illustrates specific amplification differentiation between Salmo salar & Salmo trutta, using a reverse primer with 1 additional nucleotide in 3′ compared with FIG. 1: 5SNTS-23R+3. Forward and reverse primer concentrations are as shown in FIG. 1.

FIG. 5. Illustrates the SalmoID method primers. The “preferred” primer pair 5SNTS23F+5SNTS23R+3 is indicated by the arrows. NOTE: All reverse primers are shown as their reverse complement.

FIG. 6. Illustrates results for the SalmoID method as described in example 2.

### DETAILED DESCRIPTION OF THE INVENTION

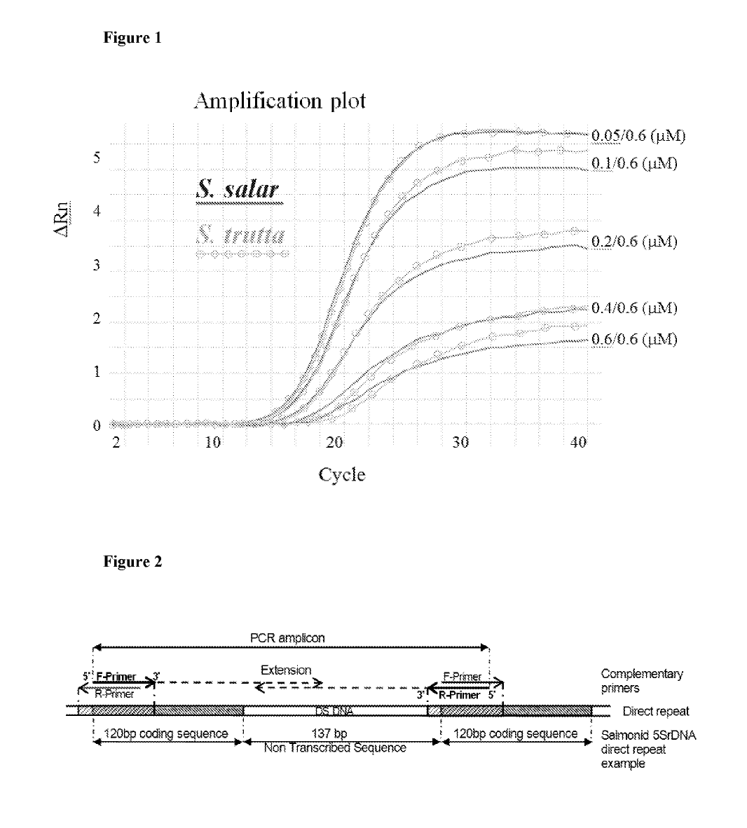

It is an object for the present invention to provide an asymmetric PCR method and non-invasive methods applicable for example for identification of salmonid species, preferably Salmo salar and Salmo trutta and their hybrids using almost complementary primers targeting direct tandem repeats. The present invention also encompasses oligonucleotide primers corresponding to species specific sequences or single nucleotide polymorphism (SNP). The use of the method and primers are also an aspect of the present invention together with kits comprising said primers. In order to unlock complementary primers for target product amplification, an asymmetric PCR method was developed by decreasing either the forward or the reverse primer concentration until optimal PCR amplification is reached. As shown in FIG. 1, this has progressively generated strong and equal amplification (unspecific) for both S. salar and S. trutta as the forward primer concentration was decreased. Asymmetric PCR has been previously described for enhancing probe based detection during which the PCR will shift from exponential to linear amplification to favor probe hybridization to its target single stranded sequence, a method called Linear-After-The-Exponential (LATE)-PCR [17, 18]. In the present invention, where the primers used are highly complementary, the asymmetric PCR has an opposite pattern shifting from linear to exponential amplification, effectively alleviating the target amplification inhibition otherwise observed with complementary primers (see FIG. 1). During the first amplification cycles the concentration-limited primer will be mainly sequestered by the excess primer such that mostly excess primer linear amplification will take place. As linear product accumulates, target sequence concentration for the limiting primer increases, sequestering excess complementary primer concentration decreases therefore enhancing target priming for the limiting primer and subsequent exponential amplification. We refer to said PCR method as COMplementary-Primer-ASymmetric (COMPAS)-PCR. The given salmonid examples (Example 2) use complementary primers which also have the same target sequence, but it is foreseen that the method may be applied to partly complementary primers having distinct target sequences.

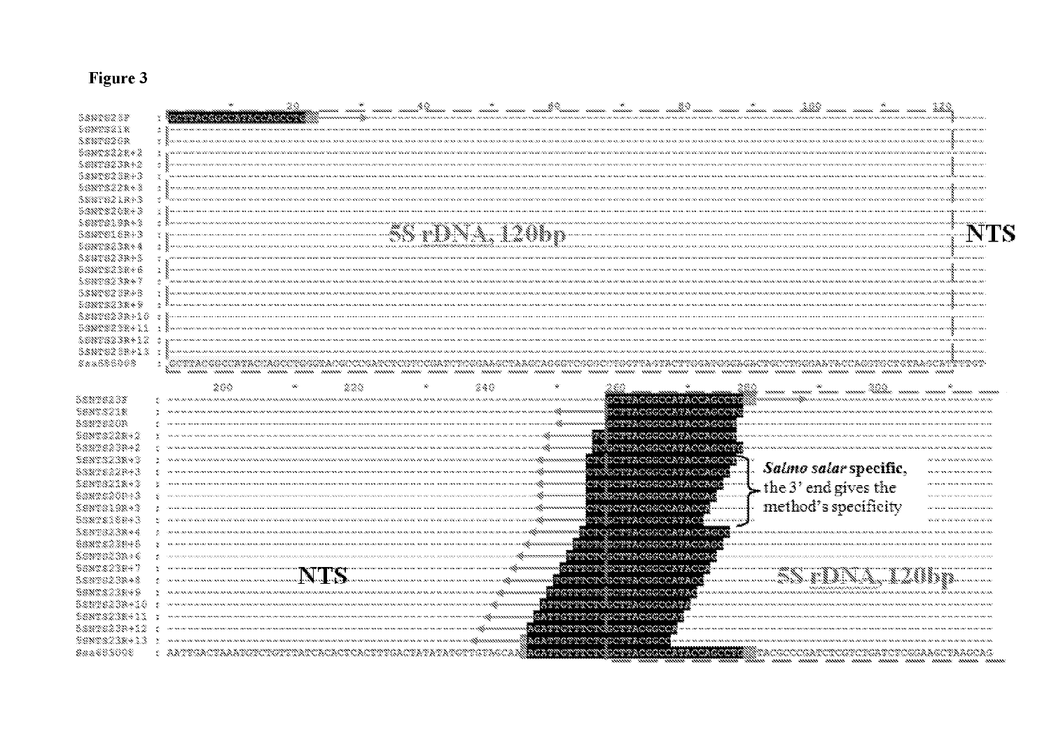

In order to identify nucleotide sequences suitable as a target for discerning between the species, the inventor made a structural study of the 5 S-rDNA tandem direct repeats and noticed that any single section of the tandem direct repeat, covering a length typical for primers (i.e. 20 bp), would be appropriate to design complementary primers structurally covering one or more products depending on the tandem direct repeat number (see FIG. 2). It is a common understanding in the art, that when designed on non-tandem direct repeats DNA, complementary primers would amplify in opposite direction, failing to define and amplify a product. In addition, complementary primer pair priming will occur and compete with primer target priming and hence strongly inhibit target amplification.

To favor priming to the target DNA contra priming to the reverse primer, the inventor increased the forward 3′ primer end by 2 nucleotides which happened to be GG (strong priming). Similarly the 3′ end of the reverse primer was extended, to favor its priming to the target contra priming to the reverse primer.

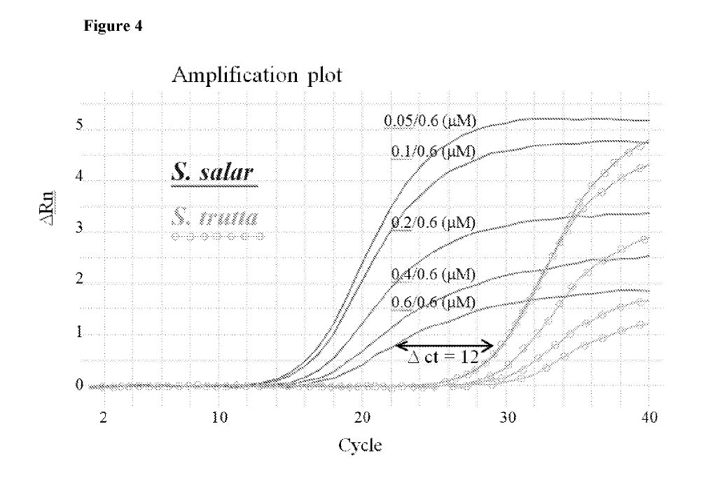

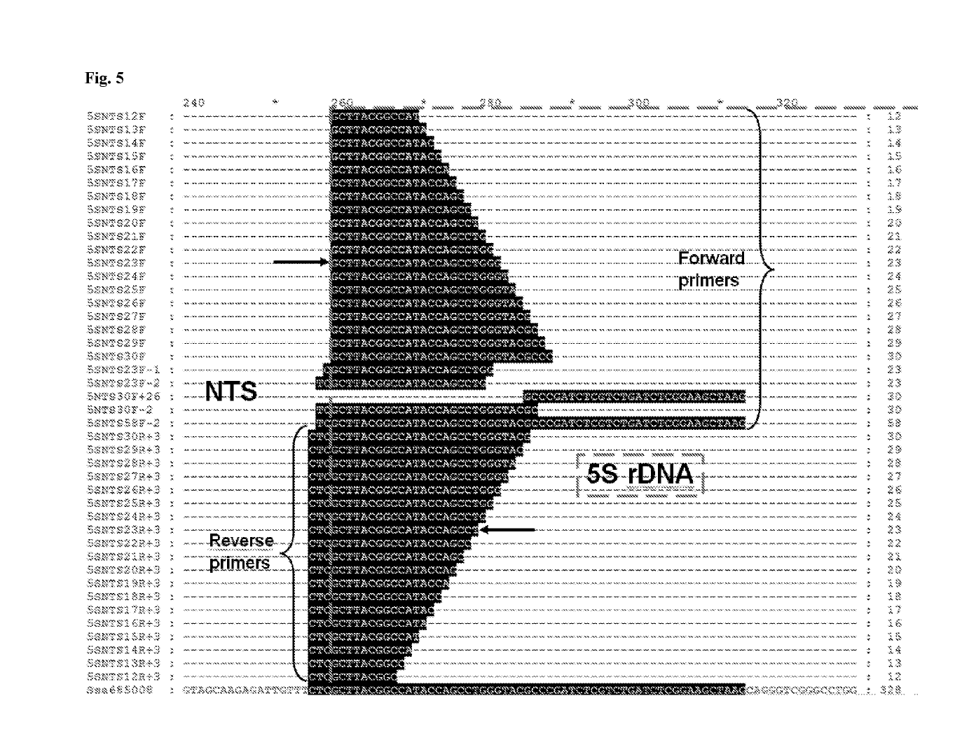

The presence of genetic variations and its adequate exploitation is the key for developing a specific test. As published sequences did not provide appropriate genetic variation information for S. salar and S. trutta, the inventor systematically tested out reverse primers, incrementing by 1 nucleotide at the 3′ end. This explored one by one the possibility for SNP. As a basis for the testing, the reverse primer was chosen, as this primer extended in the non-transcribed sequence (NTS), which is more prone to variations than the coding sequence in which the forward sequence was extending (See FIGS. 2 and 3). Following this modus operandi, the inventor observed that by extending the reverse primer in 3′ by 3 nucleotides into the NTS produced marked assay specificity with strong S. salar amplification and very weak S. trutta amplification (See FIG. 4). This specificity was conserved when the 5′ end was shortened or elongated keeping the specific 3′ “anchor” unchanged (See FIG. 3). The observed residual amplification for S. trutta was further eliminated by increasing PCR stringency (higher annealing temperature, shorter amplification time and 2 step instead of 3 step PCR). By using this approach the inventor developed a robust method and solved the problem of discerning between the Salmonids preferably Salmo salar and Salmo trutta and their hybrids, using the asymmetric PCR method according to the present invention, and almost complementary primers targeting 5S rDNA tandem direct repeats. The inventor has by using complementary primers also defeated a technical prejudice in the art [19,20], as it has been shown by the present invention that complementary primers may be used as a valuable tool when applying the COMPAS-PCR method according to the present invention.

The method is not restricted to identification of fish species. Using almost fully complementary primers targeting the same sequence may also be applicable to any organism with tandem direct repeats DNA motifs of interest as target sequences. For example the 5S r-DNA tandem direct repeats is in essence found in all eukaryotic cells [21, 22] and could therefore be further used to develop specific complementary-primer assays for other taxon and species than salmonids. Further, the method will be useful for developing assays using primers targeting distinct sequences but generating primer-dimers due to part complementarity.

Accordingly a first aspect of the present invention relates to a method of asymmetric PCR comprising:
providing a nucleic acid sample to be used as a target template;
identifying target template(s)
performing a polymerase chain reaction, utilizing highly complementary primers wherein either the forward or the reverse primer concentration is decreased to unblock the PCR reaction by initially promoting linear amplification which will progressively shift towards exponential amplification by the COMplementary-Primer-ASymmetric (COMPAS)-PCR;
identifying the amplified nucleotide target sequence(s);

In one or more embodiments the highly complementary primers may have a common overlapping DNA target sequence, said sequence may be direct tandem repeats, the direct tandem repeats target may be in the 5S-rDNA region. Other regions may however also be an option.

As used herein “complementary primers” refers to primers that are complementary to each other and will under previously known conditions bind to each other to form primer-dimers.

A second aspect of the present invention relates to a method for detecting, identification or monitoring salmonid species comprising:
providing a nucleic acid sample from salmonid to be used as (a) target template(s);
performing a polymerase chain reaction (PCR) applying COMplementary-Primer-ASymmetric (COMPAS)-PCR of the present invention, to amplify a nucleic acid target sequence of the template (s), utilizing a set or several sets of highly complementary primer pair(s) capable of priming said target(s);
identifying the amplified nucleotide target sequence(s);
determining the species.

As used herein “salmonid” refers to a family of ray-finned fish. It includes salmon, trout, chars, freshwater whitefishes and graylings. The Atlantic salmon and trout of genus Salmo give the family and order their names.

In a further embodiment the determination of the species may be performed by a melting curve analysis of the PCR product or by electrophoresis analysis. Also other methods for determination of the PCR product may be employed.

In one or more embodiments the forward primer may be extended in the 3′ end to favor priming to the target and not to the reverse primer. The reverse primer may be extended in the 3′ end to favor priming to the target and not to the forward primer. The reverse or the forward primer may have a SNP at its 3′ end. Said primer may also be slipping out to the “right” in the 5S-rDNA coding sequence out of the complementary area with the reverse primer. Further the reverse primer may be extended in the 5′ end. The 3′ forward primer may be extended by 4, preferably by 3, more preferably by 2 nucleotides.

In a further embodiment primers in the primer pair (s) are oligonucleotides each having a length of about 12 to about 30, preferably about 20 bp.

In a further embodiment the complementary set of primers may be selected from a set of primer pair (s), wherein the forward primer may be selected from Table 1 or a complementary sequence thereof and the reverse primer may be selected from Table 2 or a complementary sequence thereof or any combinations thereof.

The reverse primer may be locked at its 3′ end at “+3” in the non-coding sequence, out of the complementary forward primer area, as the specificity lies in precise positioning of this SNP. At the other end, in 5′, the reverse primer may be shorter or longer (depends on the forward primer).

In one embodiment the salmonid of the present method may be Salmo trutta, Salmo solar and hybrids thereof. The method may however, be applicable to other salmonid species, other fish species or in fact any organism with tandem repeats DNA motifs.

In a third aspect the present invention comprises oligonucleotide primers, which may be selected from the oligonucleotides of Tables 1 and 2 or any combinations thereof, or oligonucleotides with complementary sequences or functional equivalent sequences. The use of the oligonucleotides in a product, e.g a kit form furthers aspects of the present invention.

In a fourth aspect the present invention provides a kit for detecting and identification of salmonid species, comprising a collection of oligonucleotide primer pairs selected from Tables 1 and 2, in any combinations, or complementary sequences thereof, capable of detecting salmonid species by the method of the present invention.

Finally in further aspects the present invention comprises use of the methods of the present invention, use the oligonucleotide primers of the present invention or use of the kits provided from the present invention.

Having now fully described the present invention in some detail by way of illustration and example for purpose of clarity of understanding, it will be obvious to one of ordinary skill in the art that same can be performed by modifying or changing the invention by using a wide and equivalent range of conditions and other parameters thereof, and that such modifications or changes are intended to be encompassed within the scope of the appended claims.

### EXAMPLES

### Example 1

### COMPAS-PCR

Finn clips were collected from 1 S. trutta and 1 S. salar fish individuals for analysis. The samples were conserved in 98% EtOH prior to DNA extraction that was performed using mechanical and chemical methods for releasing PCR-grade DNA. Subsequent DNA measurement was performed using a nanodrop instrument (Thermo Scientific) and all samples were diluted in water to achieve a final concentration of 4 ng/μl. Samples (2.5 μl) were amplified in 25 μl final reaction mixtures using an ABI 7500 qPCR machine (Life technologies, Applied Biosystems). The mixture contained 12.5 μl MESA Blue qPCR MasterMix (Eurogentec), 0.6 μM reverse primer 5SNTS-23R+3, the forward primer 5SNTS-23F concentration was successively tested using: 0.6, 0.4, 0.2, 0.1 and 0.05 μM, the reaction was completed with distilled water, final volume of 25 μl. The 3-step PCR conditions consisted of 5 min activation at 95° C. followed by 30 cycles of 95° C. for 20 s, 62° C. for 30 s and 72° C. for 60 s. Real time amplification curves are shown in FIG. 1.

### Example 2

### Identification of Species

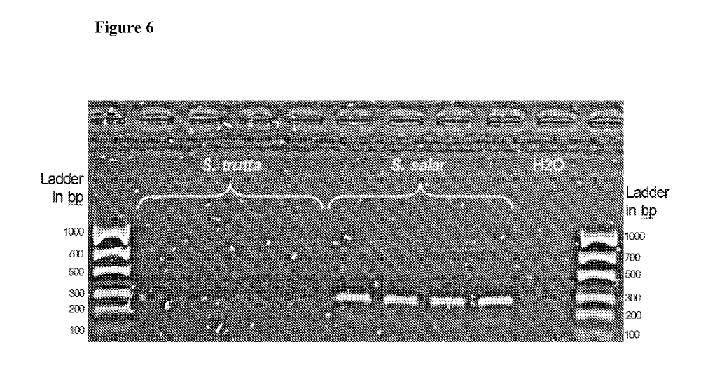

Finn clips were collected from 4 S. trutta and 4 S. salar fish individuals for analysis. The samples were conserved in 98% EtOH prior to DNA extraction that was performed using mechanical and chemical methods for releasing PCR-grade DNA. Subsequent DNA measurement was performed using a nanodrop instrument (Thermo Scientific) and all samples were diluted in water to achieve a final concentration of 4 ng/μl. Samples (2.5 μl) were amplified in 25 μl final reaction mixtures using an ABI 7500 qPCR machine (Life technologies, Applied Biosystems). The mixture contained 12.5 μl MESA Blue qPCR MasterMix (Eurogentec), 0.01 μM forward primer 5SNTS-23F, 0.6 μM reverse primer 5SNTS-23R+3 completed with distilled water. The 2-step PCR conditions consisted of 5 min activation at 95° C. followed by 30 cycles of 95° C. for 20 s and 72° C. for 30 s. Products were visualized on 1,2% agarose gels (Fermentas) stained with SybrGreen (See FIG. 6).

### REFERENCES

1. Mullis, K., et al., Specific Enzymatic Amplification of DNA Invitro—the Polymerase Chain-Reaction. Cold Spring Harbor Symposia on Quantitative Biology, 1986. 51: p. 263-273.
2. Newton, C. R., et al., Analysis of any point mutation in DNA. The amplification refractory mutation system (ARMS). Nucleic Acids Res, 1989. 17(7): p. 2503-16.
3. Bottema, C. D. and S. S. Sommer, PCR amplification of specific alleles: rapid detection of known mutations and polymorphisms. Mutat Res, 1993. 288(1): p. 93-102.
4. Sommer, S. S., et al., A novel method for detecting point mutations or polymorphisms and its application to population screening for carriers of phenylketonuria. Mayo Clin Proc, 1989. 64(11): p. 1361-72.
5. Liu, Q., et al., Overlapping PCR for Bidirectional PCR Amplification of Specific Alleles: A Rapid One-Tube Method for Simultaneously Differentiating Homozygotes and Heterozygotes. Genome Research, 1997. 7(4): p. 389-398.
6. Cha, R. S., et al., Mismatch amplification mutation assay (MAMA): application to the c-H-ras gene. PCR Methods Appl, 1992. 2(1): p. 14-20.
7. Glaab, W. E. and T. R. Skopek, A novel assay for allelic discrimination that combines the fluorogenic 5′ nuclease polymerase chain reaction (TaqMan) and mismatch amplification mutation assay. Mutat Res, 1999. 430(1): p. 1-12.
8. Birdsell, D. N., et al., Melt analysis of mismatch amplification mutation assays (Melt MAMA): a functional study of a cost-effective SNP genotyping assay in bacterial models. PLoS One, 2012. 7(3): p. e32866.
9. Urke, H., et al., Seawater tolerance in Atlantic salmon, Salmo salar L., brown trout, Salmo trutta L., and S. salar×S. trutta hybrids smolt. Fish Physiology and Biochemistry, 2010. 36(4): p. 845-853.
10. Carrera, E., et al., Identification of smoked Atlantic salmon (Salmo salar) and rainbow trout (Onchorhynchus mykiss) using PCR-restriction fragment length polymorphism of the p53 gene. Journal of Aoac International, 2000. 83(2): p. 341-346.
11. Kusser, W. C., R. L. Parker, and X. L. Miao, Polymerase Chain-Reaction and DNA-Sequence of Rainbow-Trout Tumor-Suppressor Gene-P53 Exon-5, Exon-6and Exon-7 to Exon-9. Aquatic Living Resources, 1994. 7(1): p. 11-16.
12. Dalvin, S., et al., Forensic identification of severely degraded Atlantic salmon (Salmo salar) and rainbow trout (Oncorhynchus mykiss) tissues. Investigative Genetics, 2010. 1(1): p. 12.
13. Rasmussen, R. S., M. T. Morrissey, and J. Walsh, Application of a PCR-RFLP Method to Identify Salmon Species in US Commercial Products. Journal of Aquatic Food Product Technology, 2010. 19(1): p. 3-15.
14. Pendas, A. M., et al., Chromosomal mapping and nucleotide sequence of two tandem repeats of Atlantic salmon 5S rDNA. Cytogenetic and Genome Research, 1994. 67(1): p. 31-36.
15. Komiya, H., et al., Determination of nucleotide sequence of 5S ribosomal RNA from rainbow trout liver by high performance liquid chromatography. Nucl. Acids Res., 1978. 1(suppl_{—}2): p. s467-472.
16. Pendas, A. M., et al., APPLICATIONS OF 5S-RDNA IN ATLANTIC SALMON, BROWN TROUT, AND IN ATLANTIC SALMON X BROWN TROUT HYBRID IDENTIFICATION. Molecular Ecology, 1995. 4(2): p. 275-276.
17. Wangh, L. J., et al., LATE PCR, WIPO, Editor. 2003, BRANDEIS UNIVERSITY (415 South Street Waltham, Mass., 02454-9110, US); Wangh, Lawrence J. (20 Duffield Road Auburndale, Mass., 02466, US); Pierce, Kenneth (52 Walnut Street Natick, Mass., 01760, US); Hartshorn, Cristina (1560 Great Plain Avenue Needham, Mass., 02492, US); Rice, John (268 Common Street Quincy, Mass., 02169, US); Sanchez, Aquiles J. (14 Foster Drive Framingham, Mass., 01701, US).
18. Sanchez, J. A., et al., Linear-After-The-Exponential (LATE)-PCR: An advanced method of asymmetric PCR and its uses in quantitative real-time analysis. Proceedings of the National Academy of Sciences of the United States of America, 2004. 101(7): p. 1933-1938.
19. Dieffenbach, C. W., T. M. J. Lowe, and G. S. Dveksler, General Concepts for Pcr Primer Design. Pcr-Methods and Applications, 1993. 3(3): p. S30-S37.
20. Degen, H. J., et al., PCR Applications Manual. 3d ed. 2006: Roche Diagnostics. 337.
21. Barciszewska, M. Z., et al., Structure and functions of 5S rRNA. Acta Biochimica Polonica, 2001. 48(1): p. 191-198.
22. Richard, G.-F., A. Kerrest, and B. Dujon, Comparative Genomics and Molecular Dynamics of DNA Repeats in Eukaryotes. Microbiology and Molecular Biology Reviews, 2008. 72(4): p. 686-727.

TABLE 1
Salmonid specie identification;
forward primers all indicated in 5′ to 3′

| | | SEQ |
|---|---|---|
| Name | Sequence | ID NO: |
| 5SNTS12F | GCTTACGGCCAT | 1 |
| 5SNTS13F | GCTTACGGCCATA | 2 |
| 5SNTS14F | GCTTACGGCCATAC | 3 |
| 5SNTS15F | GCTTACGGCCATACC | 4 |
| 5SNTS16F | GCTTACGGCCATACCA | 5 |
| 5SNTS17F | GCTTACGGCCATACCAG | 6 |
| 5SNTS18F | GCTTACGGCCATACCAGC | 7 |
| Ssa5SNTS19F | GCTTACGGCCATACCAGCC | 8 |
| 5SNTS20F | GCTTACGGCCATACCAGCCT | 9 |
| 5SNTS21F | GCTTACGGCCATACCAGCCTG | 10 |
| 5SNTS22F | GCTTACGGCCATACCAGCCTGG | 11 |
| 5SNTS23F | GCTTACGGCCATACCAGCCTGGG | 12 |
| 5SNTS24F | GCTTACGGCCATACCAGCCTGGGT | 13 |
| 5SNTS25F | GCTTACGGCCATACCAGCCTGGGTA | 14 |
| 5SNTS26F | GCTTACGGCCATACCAGCCTGGGTAC | 15 |
| 5SNTS27F | GCTTACGGCCATACCAGCCTGGGTACG | 16 |
| 5SNTS28F | GCTTACGGCCATACCAGCCTGGGTACGC | 17 |
| 5SNTS29F | GCTTACGGCCATACCAGCCTGGGTACGCC | 18 |
| 5SNTS30F | GCTTACGGCCATACCAGCCTGGGTACGCCC | 19 |
| 5SNTS23F − 1 | CGCTTACGGCCATACCAGCCTGG | 20 |
| 5SNTS23F − 2 | TCGCTTACGGCCATACCAGCCTG | 21 |
| 5NTS30F + 26 | GCCCGATCTCGTCTGATCTCGGAAGCTAAG | 22 |
| 5NTS30F − 2 | TCGCTTACGGCCATACCAGCCTGGGTACGC | 23 |
| 5SNTS58F − 2 | TCGCTTACGGCCATACCAGCCTGGGTACGC | 24 |
| | CCGATCTCGTCTGATCTCGGAAGCTAAG | |

TABLE 2
Salmonid specie identification;
reverse primers all indicated in 5′ to 3′

| | | SEQ |
|---|---|---|
| Name | Sequence | ID NO: |
| 5SNTS30R + 3 | CGTACCCAGGCTGGTATGGCCGTAAGCGAG | 25 |
| 5SNTS29R + 3 | GTACCCAGGCTGGTATGGCCGTAAGCGAG | 26 |
| 5SNTS28R + 3 | TACCCAGGCTGGTATGGCCGTAAGCGAG | 27 |
| 5SNTS27R + 3 | ACCCAGGCTGGTATGGCCGTAAGCGAG | 28 |
| 5SNTS26R + 3 | CCCAGGCTGGTATGGCCGTAAGCGAG | 29 |
| 5SNTS25R + 3 | CCAGGCTGGTATGGCCGTAAGCGAG | 30 |
| 5SNTS24R + 3 | CAGGCTGGTATGGCCGTAAGCGAG | 31 |
| 5SNTS23R + 3 | AGGCTGGTATGGCCGTAAGCGAG | 32 |
| 5SNTS22R + 3 | GGCTGGTATGGCCGTAAGCGAG | 33 |
| 5SNTS21R + 3 | GCTGGTATGGCCGTAAGCGAG | 34 |
| 5SNTS20R + 3 | CTGGTATGGCCGTAAGCGAG | 35 |
| 5SNTS19R + 3 | TGGTATGGCCGTAAGCGAG | 36 |
| 5SNTS18R + 3 | GGTATGGCCGTAAGCGAG | 37 |
| 5SNTS17R + 3 | GTATGGCCGTAAGCGAG | 38 |
| 5SNTS16R + 3 | TATGGCCGTAAGCGAG | 39 |
| 5SNTS15R + 3 | ATGGCCGTAAGCGAG | 40 |
| 5SNTS14R + 3 | TGGCCGTAAGCGAG | 41 |
| 5SNTS13R + 3 | GGCCGTAAGCGAG | 42 |
| 5SNTS12R + 3 | GCCGTAAGCGAG | 43 |

1. A method of asymmetric PCR comprising:
providing a nucleic acid sample to be used as a target template
identifying target template (s)
performing a polymerase chain reaction, utilizing highly complementary primers wherein either the forward or the reverse primer concentration is decreased to unblock the PCR reaction by initially promoting linear amplification which will progressively shift towards exponential amplification by the COMplementary-Primer-ASymmetric (COMPAS)-PCR;
identifying the amplified nucleotide target sequence(s).

2. The method of claim 1 wherein the highly complementary primers have a common overlapping DNA target sequence.

3. The method of claim 2 wherein the overlapping DNA target sequence is direct tandem repeats.

4. The method of claim 3 wherein the direct tandem repeat target is in the 5S-rDNA region.

5. A method for detecting, identification or monitoring salmonid species comprising:
providing a nucleic acid sample from salmonid to be used as (a) target template(s);
performing a polymerase chain reaction (PCR) applying COMplementary-Primer-ASymmetric (COMPAS)-PCR according to claim 1, to amplify a nucleic acid target sequence of the template (s), utilizing a set or several sets of highly complementary primer pair(s) capable of priming said target(s);
identifying the amplified nucleotide target sequence(s);
determining the species.

6. The method of claim 5, wherein the determination of the species is performed by a melting curve analysis of the PCR product or by electrophoresis analysis.

7. The method of claim 2, wherein the forward primer is extended in the 3′ end to favor priming to the target and not to the reverse primer.

8. The method of claim 2 wherein the reverse primer is extended in the 3′ end to favor priming to the target and not to the forward primer.

9. The method of claim 1, wherein either the reverse or the forward primer has a SNP at its 3′ end.

10. The method of claim 1, wherein the primers in the primer pair are oligonucleotides each having a length of about 12 to about 30, preferably about 20 bp.

11. The method of claim 1, wherein the complementary set of primers is selected from a set of primer pair (s), wherein the forward primer is selected from Table 1 or a complementary sequence thereof and the reverse primer is selected from Table 2 or a complementary sequence thereof.

12. The method of claim 5, wherein said salmonid comprises Salmo trutta, Salmo salar and hybrids thereof.

13. Oligonucleotide primer pairs, selected from the oligonucleotides of Tables 1 and 2 or oligonucleotides with complementary sequences or functional equivalent sequences.

14. Kit for detecting and identification of salmonid species, comprising a collection of oligonucleotide primer pairs selected from Tables 1 and 2 or complementary sequences thereof, in any combinations, capable of detecting salmonid species by the method of claim 5.

15. Use of a method of claim 1.

16. Use of the oligonucleotide primer pairs of claim 13.

17. Use of the kit of claim 14.


SEQUENCE LISTING (43 sequences)

SEQ ID NO: 1 - DNA, 12 bp, Artificial Sequence
  Oligonucleotide
gcttacggccat

SEQ ID NO: 2 - DNA, 13 bp, Artificial Sequence
  Oligonucleotide
gcttacggccata

SEQ ID NO: 3 - DNA, 14 bp, Artificial Sequence
  Oligonucleotide
gcttacggccatac

SEQ ID NO: 4 - DNA, 15 bp, Artificial Sequence
  Oligonucleotide
gcttacggccatacc

SEQ ID NO: 5 - DNA, 16 bp, Artificial Sequence
  Oligonucleotide
gcttacggccatacca

SEQ ID NO: 6 - DNA, 17 bp, Artificial Sequence
  Oligonucleotide
gcttacggccataccag

SEQ ID NO: 7 - DNA, 18 bp, Artificial Sequence
  Oligonucleotide
gcttacggccataccagc

SEQ ID NO: 8 - DNA, 19 bp, Artificial Sequence
  Oligonucleotide
gcttacggccataccagcc

SEQ ID NO: 9 - DNA, 20 bp, Artificial Sequence
  Oligonucleotide
gcttacggccataccagcct

SEQ ID NO: 10 - DNA, 21 bp, Artificial Sequence
  Oligonucleotide
gcttacggccataccagcctg

SEQ ID NO: 11 - DNA, 22 bp, Artificial Sequence
  Oligonucleotide
gcttacggccataccagcctgg

SEQ ID NO: 12 - DNA, 23 bp, Artificial Sequence
  Oligonucleotide
gcttacggccataccagcctggg

SEQ ID NO: 13 - DNA, 24 bp, Artificial Sequence
  Oligonucleotide
gcttacggccataccagcctgggt

SEQ ID NO: 14 - DNA, 25 bp, Artificial Sequence
  Oligonucleotide
gcttacggccataccagcctgggta

SEQ ID NO: 15 - DNA, 26 bp, Artificial Sequence
  Oligonucleotide
gcttacggccataccagcctgggtac

SEQ ID NO: 16 - DNA, 27 bp, Artificial Sequence
  Oligonucleotide
gcttacggccataccagcctgggtacg

SEQ ID NO: 17 - DNA, 28 bp, Artificial Sequence
  Oligonucleotide
gcttacggccataccagcctgggtacgc

SEQ ID NO: 18 - DNA, 29 bp, Artificial Sequence
  Oligonucleotide
gcttacggccataccagcctgggtacgcc

SEQ ID NO: 19 - DNA, 30 bp, Artificial Sequence
  Oligonucleotide
gcttacggccataccagcctgggtacgccc

SEQ ID NO: 20 - DNA, 23 bp, Artificial Sequence
  Oligonucleotide
cgcttacggccataccagcctgg

SEQ ID NO: 21 - DNA, 23 bp, Artificial Sequence
  Oligonucleotide
tcgcttacggccataccagcctg

SEQ ID NO: 22 - DNA, 30 bp, Artificial Sequence
  Oligonucleotide
gcccgatctcgtctgatctcggaagctaag

SEQ ID NO: 23 - DNA, 30 bp, Artificial Sequence
  Oligonucleotide
tcgcttacggccataccagcctgggtacgc

SEQ ID NO: 24 - DNA, 58 bp, Artificial Sequence
  Oligonucleotide
tcgcttacggccataccagcctgggtacgcccgatctcgtctgatctcggaagctaag

SEQ ID NO: 25 - DNA, 30 bp, Artificial Sequence
  Oligonucleotide
cgtacccaggctggtatggccgtaagcgag

SEQ ID NO: 26 - DNA, 29 bp, Artificial Sequence
  Oligonucleotide
gtacccaggctggtatggccgtaagcgag

SEQ ID NO: 27 - DNA, 28 bp, Artificial Sequence
  Oligonucleotide
tacccaggctggtatggccgtaagcgag

SEQ ID NO: 28 - DNA, 27 bp, Artificial Sequence
  Oligonucleotide
acccaggctggtatggccgtaagcgag

SEQ ID NO: 29 - DNA, 26 bp, Artificial Sequence
  Oligonucleotide
cccaggctggtatggccgtaagcgag

SEQ ID NO: 30 - DNA, 25 bp, Artificial Sequence
  Oligonucleotide
ccaggctggtatggccgtaagcgag

SEQ ID NO: 31 - DNA, 24 bp, Artificial Sequence
  Oligonucleotide
caggctggtatggccgtaagcgag

SEQ ID NO: 32 - DNA, 23 bp, Artificial Sequence
  Oligonucleotide
aggctggtatggccgtaagcgag

SEQ ID NO: 33 - DNA, 22 bp, Artificial Sequence
  Oligonucleotide
ggctggtatggccgtaagcgag

SEQ ID NO: 34 - DNA, 21 bp, Artificial Sequence
  Oligonucleotide
gctggtatggccgtaagcgag

SEQ ID NO: 35 - DNA, 20 bp, Artificial Sequence
  Oligonucleotide
ctggtatggccgtaagcgag

SEQ ID NO: 36 - DNA, 19 bp, Artificial Sequence
  Oligonucleotide
tggtatggccgtaagcgag

SEQ ID NO: 37 - DNA, 18 bp, Artificial Sequence
  Oligonucleotide
ggtatggccgtaagcgag

SEQ ID NO: 38 - DNA, 17 bp, Artificial Sequence
  Oligonucleotide
gtatggccgtaagcgag

SEQ ID NO: 39 - DNA, 16 bp, Artificial Sequence
  Oligonucleotide
tatggccgtaagcgag

SEQ ID NO: 40 - DNA, 15 bp, Artificial Sequence
  Oligonucleotide
atggccgtaagcgag

SEQ ID NO: 41 - DNA, 14 bp, Artificial Sequence
  Oligonucleotide
tggccgtaagcgag

SEQ ID NO: 42 - DNA, 13 bp, Artificial Sequence
  Oligonucleotide
ggccgtaagcgag

SEQ ID NO: 43 - DNA, 12 bp, Artificial Sequence
  Oligonucleotide
gccgtaagcgag